(19) United States
(12) Patent Application Publication
(10) Pub. No.: US 2006/0122669 A1
(43) Pub. Date: Jun. 8, 2006

(54) Method and apparatus for insulating body organs during transplantation

(76) Inventors: Hassan A. Kansoul, Stockholm (SE); Daniel K. Farley, Traverse City, MI (US); Anthony J. Mulac, Traverse City, MI (US)

Correspondence Address: MCANDREWS HELD & MALLOY, LTD, 500 WEST MADISON STREET, SUITE 3400, CHICAGO, IL, 60661, US

(21) Appl. No.: 10/972,758

(22) Filed: Oct. 25, 2004

(51) Int. Cl. A61F 7/00; A61F 7/12

(52) U.S. Cl. 607/96; 607/112; 607/113

(57) ABSTRACT

A method and apparatus for thermally affecting graft organs during harvesting and transplantation operations. An insulation jacket is configured to conform to the shape of the graft organ. The insulation jacket includes a body portion constructed from a flexible surgical grade plastic, insulation foam, or thermo mass. The body portion may include a plurality of non-communicating pillows capable of retaining a malleable condition when chilled to desired implantation temperatures. The selection of material for the body portion and the sterile nature of the cooling material allow the insulation jacket to be placed inside a patient's body during surgery. When secured about a graft organ, the insulation jacket provides openings for access to graft organ vessels, thereby allowing the implantation surgery to proceed while at least a portion of the graft organ remains enclosed in the insulation jacket. The insulation jacket may also include at least one layer of insulation.

### BACKGROUND OF THE INVENTION

The present invention pertains to a method and apparatus for thermally affecting graft organs during harvesting and transplantation procedures. More particularly, the present invention pertains to an insulation jacket that may cool graft organs during harvesting procedures and may insulate the graft organ from heat sources during transplantation.

Transplantation surgery is one of the leading and fastest growing surgical technologies of our time. The rapid development of this field is due to current organ donation policies, changes in public awareness and viewpoints pertaining to the necessity for donors, and recent technical innovations that are making transplantations easier and safer to perform. While technological improvements have reduced some of the complications associated with transplantation surgery, other severe complications continue to exist.

One of the most frequent complications in transplantation surgeries is ischemia. Ischemia is the reduction or stoppage of flow of oxygen and nutrients to living cells. Ischemia may have drastic consequences, including apoptosis and senescence, which entails the death of the oxygen and nutrient deprived cells. Unfortunately, because the supply of blood and nutrients ceases when a graft organ is removed from a donor's body, ischemia is always present in transplantation procedures.

Ischemia has been the central focus of numerous research projects and studies. As a result of these studies, many theories have been developed to better explain the detection and consequences of ischemia and to suggest how to slow down its damaging effects. Previous attempts to limit the consequences of ischemia, and thereby improve graft organ preservation, include training centers for surgeons devoted to reducing operation time and improving implantation techniques, the development of drugs designed to protect cells from entering into apoptosis, and the cooling of donated graft organs prior to implantation.

From these studies and projects, two types of ischemia have been defined, namely warm ischemia and cold ischemia. Warm ischemia typically begins when the blood supply to the organ is stopped and the graft organ is removed from the donor's body. The onset of apoptosis during warm ischemia typically occurs extremely fast and leads to irreversible damages. Injury due to warm ischemia has had a severe influence on the viability and post-transplantation outcome of organ grafts. However, after many years of research, it was discovered that the apoptosis process could be slowed down by reducing the temperature of the graft organ, which is known as cold ischemia.

Cold ischemia reduces the temperature of the removed graft organ so that enzymatic activity is delayed or stops altogether. The slowing of the apoptosis process through the use of cold ischemia significantly reduces the presence of irreversible complications. For example, while exposure of the graft organ to warm ischemia is measured in minutes, cold ischemia is measured in hours. Thus, the discovery of the benefits of cold ischemia over warm ischemia was a big step towards improving the success of transplantation procedures.

Yet, injuries due to cold and warm ischemia remain an important source of morbidity and mortality in some transplantation procedures. For instance, while cold ischemia may slow the apoptosis process, the graft organ may be irreparably damaged if cooled below 4° Celsius. Further, similar to warm ischemia, there is a time limit on how long a graft organ may withstand cold ischemia.

However, because of the benefits offered by cold ischemia, graft organs are prepared for transplantation in a cold environment. A graft organ is typically reduced to a temperature of approximately 4° Celsius. However, during the implantation operation, the temperature of the graft organ gradually increases and results in the onset of undesirable warm ischemia. This increase in temperature may be facilitated by the exposure of the graft organ to a number of different heat sources, including warmth from the recipient's body, the surgeon's hands, operating lights, general room illumination, room temperature, and operating instruments.

Many existing cooling packs are designed to cool a variety of items, including food, beverages, and biological materials, such as organs. Such cooling packs may be similar to ice packs in which a sealed pouch is filled with a thermal cooling agent that is converted to a frozen solid state when cooled, whereupon the cooling agent may no longer be malleable. Other prior art devices include a thermal cooling agent that does not transform into a solid state when cooled. However, these thermal cooling agents are often contain within a single bladder-like enclosure or a series of individual chambers that are in communication with adjacent chambers. The problem with such configurations is that if one section of the bladder or a single chamber is accidentally or purposefully breached, a significant portion, if not all, of the cooling agent may flow out of the cooling pack, and thus hinder or ruin the ability of the cooling pack to function.

Further, prior art cooling packs may have a general, non-organ specific shape or configuration. Unfortunately, in organ transplantation and harvesting operations, the general shape of a cooling pack may result in unnecessary interference with the field of operation. Additionally, by not providing a cooling pack that is configured for use with a specific type of graft organ, the prior art devices may not, when at least partially secured about a graft organ, provide openings through which graft organ vessels or veins may pass away from the graft organ and cooling pack. The lack of openings may prevent the surgeon from having access to necessary vessels and veins during a harvesting or transplantation procedure.

Therefore, it is an object of the present invention to prevent or delay the warming of a graft organ during an implantation process.

It is another object of the present invention to provide an apparatus and method for insulating a graft organ placed inside a recipient's body during a transplantation operation and thereby prevent the onset of warm ischemia.

It is a further object of the present invention to provide a thin organ insulation jacket that is configured to have the general shape of the particular graft organ that is being harvested or transplanted.

It is another object of the present invention to provide an organ insulation jacket that provides a surgeon with access to required vessels of the graft organ during transplantation surgery while the insulation jacket continues to enclose at least a portion of the graft organ.

It is a further object of the present invention to provide an insulation jacket that may be sterilized so that the graft organ may be enclosed by the insulation jacket while inside a patient's body during a transplantation procedure.

It is also an object of the present invention to provide a insulation jacket that may include a non-toxic and sterile cooling material that may reduce the temperature of the graft organ, the cooling material being able to retain a malleable nature when cooled to desired implantation temperatures.

A least one of the preceding objects is met, in whole or in part, by the present invention, which will become apparent in view of the present specification, including the claims and drawings.

### BRIEF SUMMARY OF THE INVENTION

The present invention pertains to a method and apparatus for insulating graft organs during harvesting and transplantation procedures. More particularly, the present invention pertains to an insulation jacket that may enclose at least a portion of a graft organ and which may be sterilized so as to be capable of being placed within the body of a patient during a harvesting and transplantation operation. The insulation jacket includes a body portion that may be constructed from a flexible surgical grade plastic, insulation foam, or a thermo mass, for example a dense gel. The body portion may also be operably connected to connectors that allow the body portion to be secured in a closed position when manipulated about at least a portion of a graft organ. In one embodiment of the present invention, the body portion may be configured to form a plurality of pillows, the pillows being configured to contain a non-toxic sterile cooling material. In such an embodiment, the cooling material may assist in reducing the temperature of the graft organ to desired transplantation temperatures. Further, some embodiments of the present invention may also include at least one layer of insulation that may assist in insulating the graft organ and/or cooling materials from outside heat sources. The selection of materials for the body portion and, when used, the sterile nature of the cooling material allows the insulation jacket to be placed inside a patient's body during harvesting and transplantation surgery.

The body portion includes inner and outer walls and may be specifically shaped for a particular type of organ and its attached veins and vessels, such as, but not limited to, a heart, liver, lung, pancreas, and kidney. The body portion may be comprised of at least one panel. The panel may be operably connected to an adjacent panel through the use of an adhesive. The body portion may also be configured so that, when enclosed about at least a portion of the graft organ, the insulation jacket includes openings which may be positioned around vessels and arteries of the graft organ so that the vessels may pass through or extend away from the insulation jacket. These openings in the insulation jacket provide the surgeon with access to organ vessels, which allows the surgeon to conduct the transplantation surgery while at least a portion of the graft organ remains enclosed in the insulation jacket.

The non-toxic sterile cooling material may include, but is not limited to, a sterile liquid or gel. The selected cooling material may be capable of retaining a malleable condition when cooled to a temperature of approximately 4° Celsius. Each of the plurality of pillows may not be in communication with adjacent pillows, and thus the puncture or rupture of one pillow may not result in the loss of cooling material from adjacent pillows. Therefore, in the event that a pillow is punctured, torn, or ruptures, adjacent pillows may still retain the chilled or unchilled cooling material.

Further, the selection of materials for the body portion may also allow the body portion to have an elastic nature. For example, the flexible nature of some surgical grade plastics may allow at least some areas of the body portion between the non-communicating pillows to function as elastic bands. Besides connecting adjacent pillows, the flexible nature of any such elastic bands could assist in the ability of the insulation jacket to be manipulated about the specific graft organ that the insulation jacket was designed to at least partially enclose.

In some embodiments of the present invention, the body portion may be operably connected to at least one layer of insulation. The insulation may be operably affixed along at least a portion of the inner wall and/or outer wall of the body portion of the insulation jacket. Also, the layer of insulation may be constructed from a number of different materials, including, but not limited to, a closed cell insulating foam, including a polyethylene closed cell foam. Further, the layer of insulation may be thin and malleable in nature so as to not interfere with the flexibility of the body portion. The layer of insulation may also serve as a wall of the body portion.

When used, the insulation jacket may be placed around the organ before transplantation into the recipient, including prior to harvesting the graft organ from the donor's body. For example, when possible, to further attempt to minimize the onset of warm ischemia, it may be preferable to not cut the vessels supplying blood and oxygen to the harvested organ until the insulation jacket has been placed around the graft organ. Once secured around the graft organ, the graft organ may be removed from the donor's body. The graft organ may then be prepared for implantation into the body of the recipient.

While being prepared for transplantation, the organ is preferably maintained at approximately 4° Celsius. During the transplantation operation, the graft organ may remain enclosed at least in part by the insulation jacket so as to prevent damage to the cells that is associated with warm ischemia. For embodiments of the present invention that include a plurality of pillows, because the pillows may not be in communication with adjacent pillows, the loss of cooling material in pillows that are damaged (i.e. pillows that may be ruptured, punctured, tom, severed, or pierced) may not cause undamaged adjacent pillows to lose any of their cooling material. Therefore, the surgeon may accidentally or purposely remove cooling material from some pillows without completely destroying the ability of the insulation jacket to continue to cool the graft organ.

### BRIEF DESCRIPTION OF SEVERAL VIEWS OF THE DRAWINGS

FIG. 1 illustrates an inside elevation view of an insulation jacket in accordance with one embodiment of the present invention.

FIG. 2 illustrates an outside elevation view of an insulation jacket configured to enclose at least a portion of a graft organ in accordance with one embodiment of the present invention.

FIG. 3 illustrates a cross sectional view of the pillows of an insulation jacket in accordance with one embodiment of the present invention.

FIG. 4 illustrates an outside elevation view of an insulation jacket having a plurality of panels and connectors in which the insulation jacket is secured about at least a portion of a graft organ in accordance with one embodiment of the present invention.

FIG. 5 illustrates a partial, cross-sectional end view of an insulation jacket in accordance with one embodiment of the present invention.

FIG. 6 illustrates an outside elevation view of an insulation jacket having an insulated outer wall in accordance with one embodiment of the present invention.

FIG. 7 illustrates a cross sectional view of an insulation jacket having an insulated outer wall in accordance with one embodiment of the present invention.

FIG. 8 illustrates of an outside elevation view of an insulation jacket having a plurality of panels and connectors in which the insulation jacket is secured about at least a portion of a graft organ in accordance with one embodiment of the present invention.

FIG. 9 illustrates a partial, cross-sectional end view of an insulation jacket having insulation on the outer wall in accordance with one embodiment of the present invention.

FIG. 10 illustrates an inside elevation view of an insulation jacket having an insulated outer wall and an inner wall that is not insulated in accordance with one embodiment of the present invention.

FIG. 11 illustrates an outside elevation view of an insulation jacket having an insulated outer wall and an insulated inner wall in accordance with one embodiment of the present invention.

FIG. 12 illustrates a cross sectional view of the insulation jacket that is insulated on both the inner wall and outer wall in accordance with one embodiment of the present invention.

FIG. 13 illustrates of an outside elevation view of an insulation jacket having a plurality of panels and connectors in which the insulation jacket is secured about at least a portion of a graft organ in accordance with one embodiment of the present invention.

FIG. 14 illustrates a partial, cross-sectional end view of an insulation jacket having insulation on both the inner wall and outer wall in accordance with one embodiment of the present invention.

FIG. 15 illustrates an inside elevation view of insulation jacket having an insulated inner and outer wall in accordance with one embodiment of the present invention.

FIG. 16 illustrates an inside elevation view of an insulation jacket in accordance with one embodiment of the present invention in which the body portion is comprised of an insulation foam.

FIG. 17 illustrates an outside elevation view of an insulation jacket configured to enclose at least a portion of a graft organ in accordance with one embodiment of the present invention in which the body portion is comprised of an insulation foam.

FIG. 18 illustrates a cross sectional view of the closed cells of an insulation jacket in accordance with one embodiment of the present invention in which the body portion is comprised of an insulation foam.

FIG. 19 illustrates of an outside elevation view of an insulation jacket having a plurality of panels and connectors in which the insulation jacket is secured about at least a portion of a graft organ in accordance with one embodiment of the present invention in which the body portion is comprised of an insulation foam.

FIG. 20 illustrates an inside elevation view of an insulation jacket in accordance with one embodiment of the present invention in which the body portion is comprised of a thermo mass.

FIG. 21 illustrates an outside elevation view of an insulation jacket configured to enclose at least a portion of a graft organ in accordance with one embodiment of the present invention in which the body portion is comprised of a thermo mass.

FIG. 22 illustrates of an outside elevation view of an insulation jacket having a plurality of panels and connectors in which the insulation jacket is secured about at least a portion of a graft organ in accordance with one embodiment of the present invention in which the body portion is comprised of a thermo mass.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentalities shown in the attached drawings.

### DETAILED DESCRIPTION OF THE INVENTION

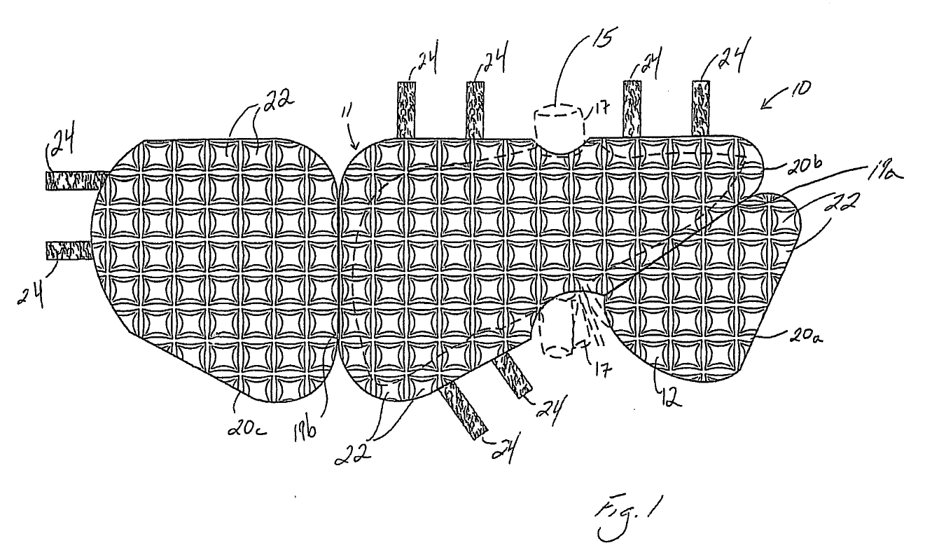

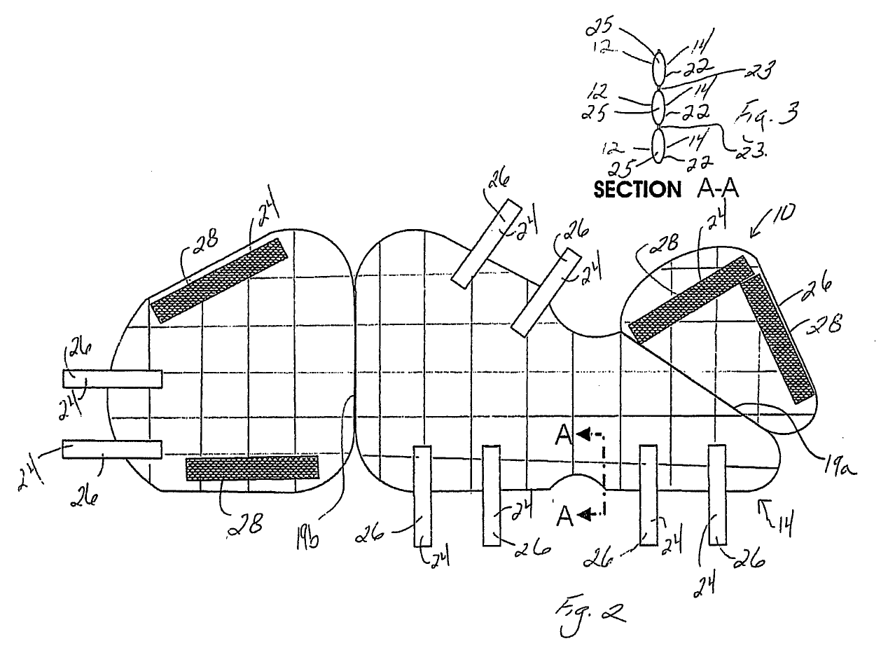

FIG. 1 illustrates an inside elevation view of an insulation jacket 10 that is configured to enclose at least a portion of a graft organ 15 in accordance with one embodiment of the present invention. The insulation jacket 10 may include a body portion 11 and a plurality of connectors. As shown in FIGS. 1 and 2, the body portion 11 has an inner wall 12 and an outer wall 14 and may be constructed from relatively thin and flexible surgical grade plastics. Further, the inner wall 12 and outer wall 14 may be separate pieces of flexible surgical grade plastics that are operably connected, such as through the use of adhesive materials, to form the body portion 11. The size and shape of the body portion 11 may be determined by the specific type of graft organ 15. More specifically, the insulation jacket 11 may be contoured and sized to enclose at least a portion of a specific organ, for example a heart, liver, lung, or kidney, while still providing access to the vessels 17 of the graft organ 15.

For example, as is known, different types of graft organs have different shapes and sizes. More specifically, although the actual size of a graft organ may vary from person to person, generally, a normal adult liver may be 28 cm long, 8 cm in height, and 18 cm in antero-posterior thickness. Further, an average kidney in a living adult may weigh from 2000 to 2500 grams, while the liver in a cadaver may weigh 1400 to 1500 grams. Although the actual dimensions of the kidney of an adult may vary depending on a variety of factors, including sex, age, and pathology, a normal adult kidney may be 10 to 12 cm long vertically, 5 to 6 cm wide, have a 3 cm antero-posterior thickness, and weigh 115 to 150 grams. Additionally, while a variety of factors may also affect the size of a lung, a lung removed from an adult of intermediary respiratory status may have a vertical length of 25 cm, a width at the base of 15 cm, a transversal base measurement of 10 cm, and a weight of 700 grams. Therefore, by designing the shape of the body portion 11 to cover at least a portion of specific type of graft organ 15, the organ specific body portion 11 may occupy a minimal amount of space in the operation field and thus may minimize the potential risk that the insulation jacket 10 may interfere with the vision or maneuverability of the surgeon during an implantation operation.

While the body portion 11 may be a single panel 20 that is designed to encompass at least a portion of a graft organ 15, in the illustrated embodiment, the body portion 11 may be comprised of a plurality of panels 20a, 20b, 20c that are operably connected to an adjacent panel. In such an embodiment, the panels 20a, 20b, 20c may be operably connected to an adjacent panel through the use of an adhesive. The location of the attachment of panels 20a, 20b, 20c may result in the formation of creases 19a, 19b between adjacent panels 20a, 20b, 20c. Because the insulation jacket 10 may be designed for use with a specific type of graft organ 15, the creases 19a, 19b may be positioned in locations in which the body portion 11 is folded about at least a portion of the graft organ, thereby assisting in preventing the formation of undesirable protruding points and corners that may harm adjacent tissue or take up additional space in the operating field.

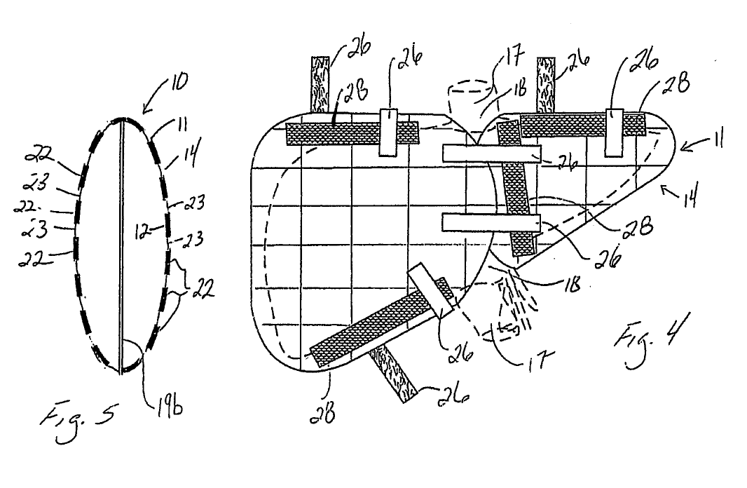

FIG. 3 illustrates a cross sectional view the pillows of an insulation jacket 10 in accordance with one embodiment of the present invention. As illustrated in FIG. 3, the inner wall 12 and outer wall 14 of the body portion 11 may be configured to allow for the formation of a plurality of pillows 22. The pillows 22 may include an inner section 25 that is configured to contain a cooling material. The cooling material may be a sterile liquid or gel that will not freeze solid when cooled to temperatures of at least approximately 4° Celsius and which will remain malleable at low temperatures. Suitable cooling material include, but are not limited to, Aquasonic™ Clear ultrasound gel from Parker Laboratories Inc. of Fairfield, N.J., lubricants such as K-Y™ lubricants offered by a division of McNeil-P.P.C., Inc. of Skillman, N.J., a Johnson and Johnson company, and saline solutions.

In the illustrated embodiment, the pillows 22 may not be in communication with other adjacent pillows 22, but instead may be separated so that the tearing or puncturing of one pillow 22 will not result in the loss of cooling material from an adjacent undamaged pillow 22. In the illustrated embodiment, the connection of the inner wall 12 and outer wall 14 across the interconnecting region between adjacent non-communicating pillows 22 allows for the formation of bands 23. Because of the flexible nature of the material used for the body portion 11, the bands 23 may be elastic in nature and thus may assist in providing flexibility and/or plasticity to the body portion 11.

FIG. 2 illustrates an outside elevation view of an insulation jacket 10 configured in accordance with one embodiment of the present invention. Connectors may be used to secure the body portion 11 around at least a portion of the graft organ 15. The connectors may include, but are not limited to, mating strips of hook and loop material, staples, tape, and adhesives.

FIG. 4 illustrates an outside elevation view of an insulation jacket 10 having a plurality of panels 20a, 20b, 20c and connectors in which the insulation jacket is secured about at least a portion of a graft organ in accordance with one embodiment of the present invention. As shown, the connectors may include mating strips of hook and loop material in which a first strip 26 is positioned to attach to a mating second strip 28 when the body portion 11 is partially secured around a graft organ 15. Further, in the embodiment of the present invention illustrated in FIGS. 1, 2, and 4, the connectors may be operably attached to the outer wall 14 of the body portion 11, including through the use of an adhesive material.

As also illustrated in FIG. 4, the contours and shape of the body portion 11 allow the insulation jacket 10 to enclose at least a portion of the graft organ 15 while still providing openings 18 for the vessels 17 and/or arteries of the graft organ 15 to pass out of the insulation jacket 10. By providing these openings 18, a surgeon may have access to the main vessels 17 and/or veins while the insulation jacket 10 is placed around at least a portion of the graft organ 15 during the removal and/or subsequent transplantation of the graft organ 15.

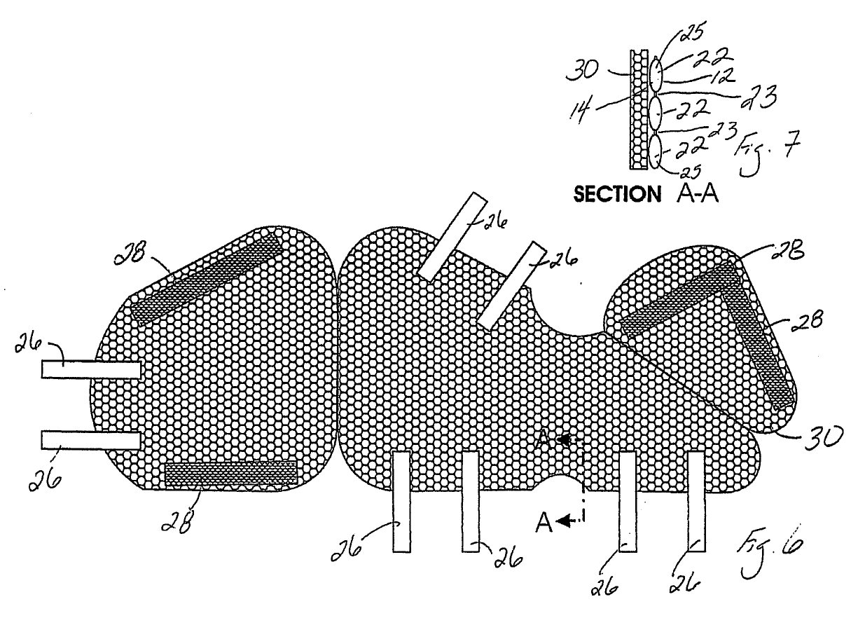

FIG. 5 illustrates a partial, cross-sectional end view of an insulation jacket 10 of FIG. 4. When wrapped about at least a portion of a graft organ (not shown), the surface of the pillows 22 along the inner wall 12 may come into direct contact with at least a portion of the enclosed graft organ. In such an embodiment, because of the potential direct physical contact between the surface of the pillows 22 along the inner wall 12 and the graft organ, the temperature of the cooling material contained within the pillows 22 should not be reduced to temperatures that may cause damage to the graft organ. More particularly, the temperature of the pillows 22 and cooling material contained therein preferably should not be cooled to be below 4° Celsius so as to ensure that the tissue and cells of the graft organ that come into contact with the pillows 22 are not unnecessarily damaged.

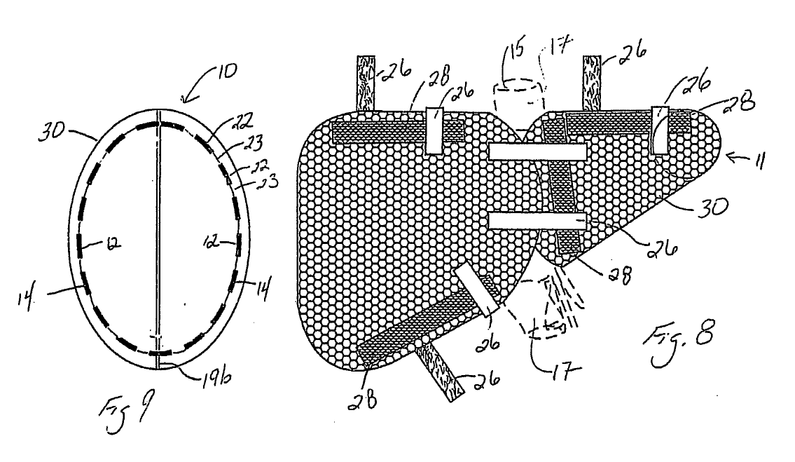

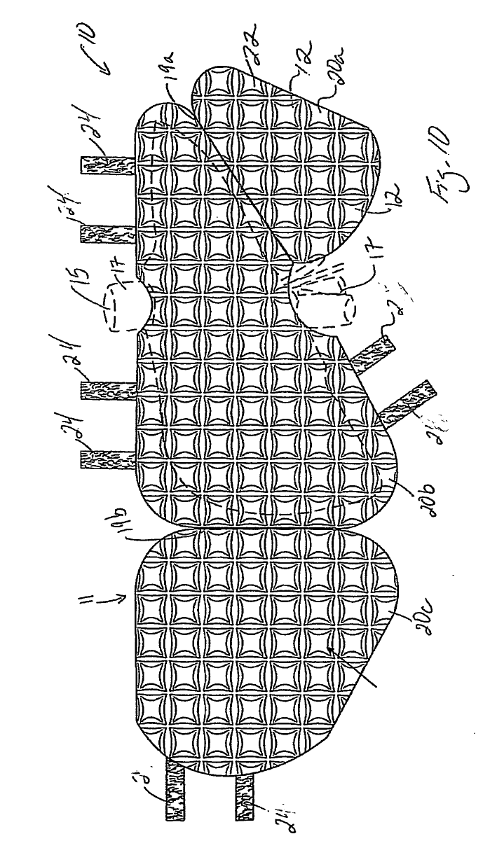

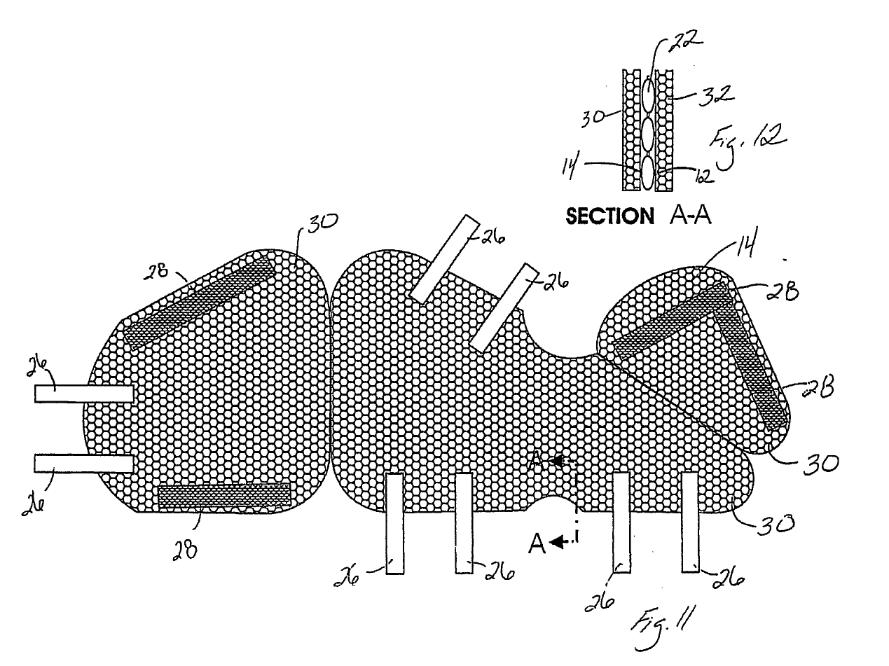

FIGS. 6, 7, 8, 9, and 10 illustrate another embodiment of the present invention in which the insulation jacket 10 illustrated in FIGS. 1-5 also includes an outer layer of insulation 30 along at least a portion of the outer wall 14. The outer layer of insulation 30 may be operably attached to the body portion 11, including through the use of an adhesive. For example, the outer layer of insulation 30 may be adhered to the outer wall 14. Further, as shown, the outer layer of insulation 30 may also be operably connected to the first and second strips 26, 28 of the connectors, such as through the use of an adhesive material. Additionally, the outer layer of insulation 30 may be comprised of an insulation foam, including, but not limited to, a closed cell insulating foam, for example a polyethylene foam. Alternatively, the outer layer of insulation 30 may be comprised of multiple layers of sterile drapes that are encapsulated in a surgical grade material. By placing the outer layer of insulation 30 along the outer wall 14, the outer layer of insulation 30 may insulate the insulation jacket 10 from outside heat sources, including, but not limited to, heat from the body of the patient, surgical lights, and surgical equipment, and thereby assist in retaining the cool temperature of the cooling material and/or graft organ 15 for longer periods of time. Similarly, the layer of insulation may be positioned along a portion of the inner wall 12 of the insulation jacket 10 rather than being along the outer wall 14, as would be understood by one of ordinary skill in the art.

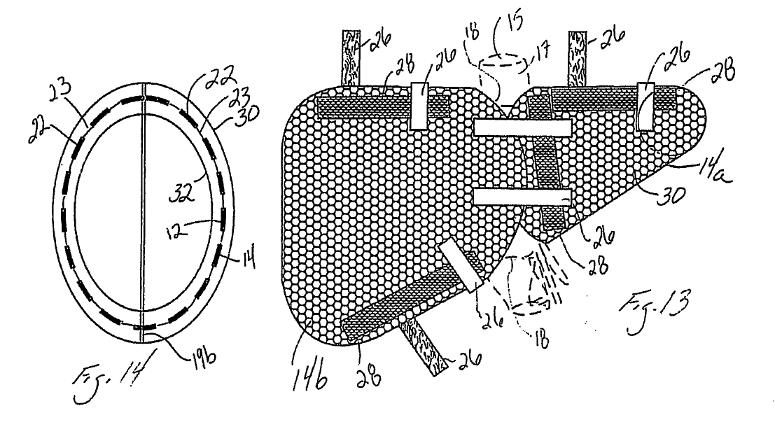

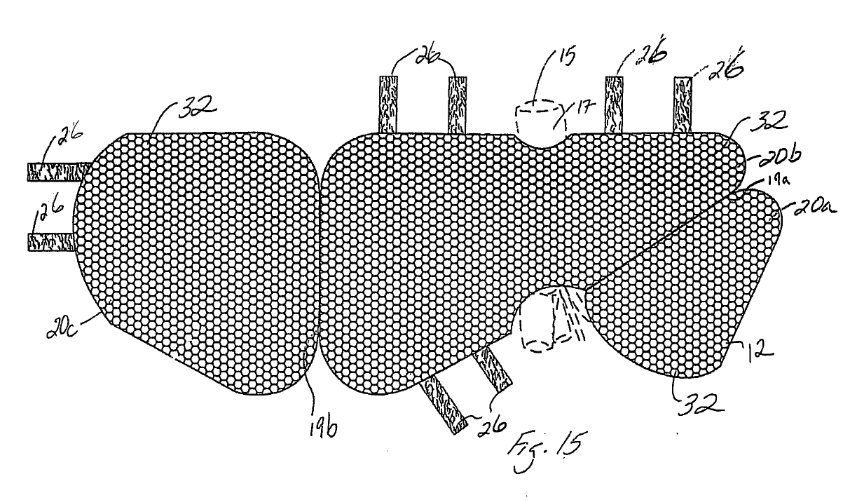

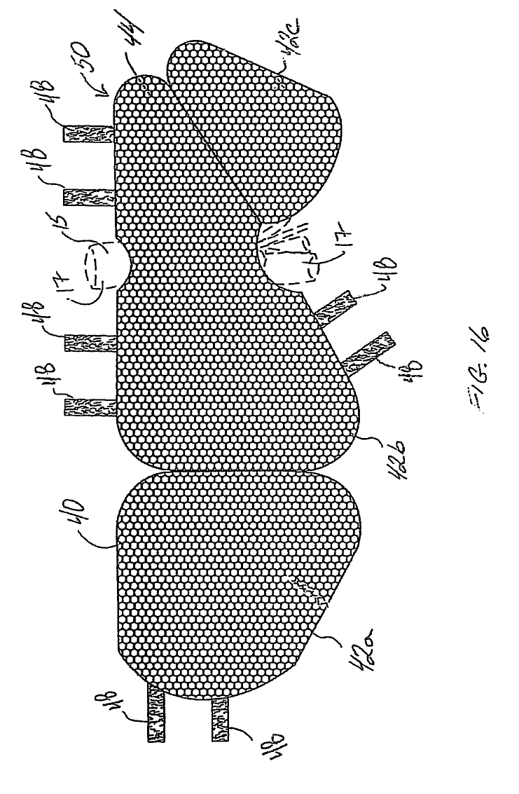

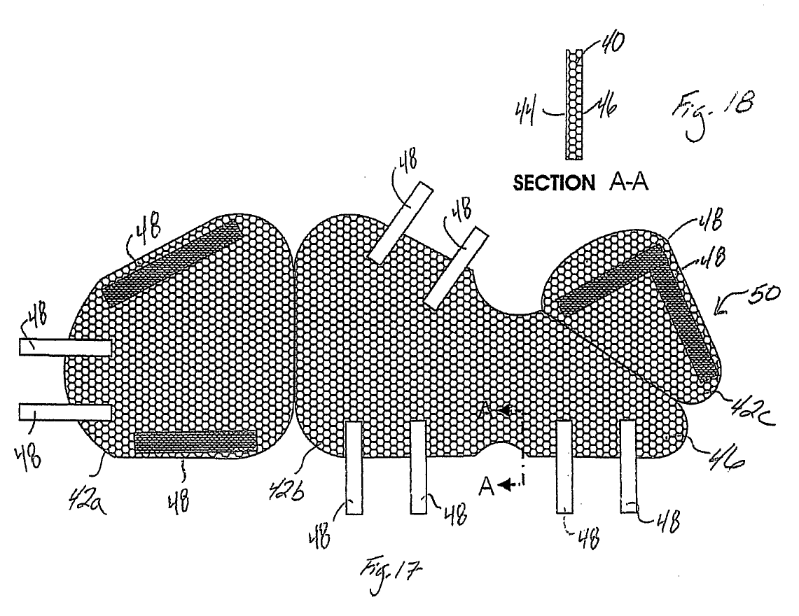

FIGS. 11, 12, 13, 14, and 15 illustrate yet another embodiment of the insulation jacket 10 illustrated in FIGS. 1-5 in which both the inner and outer walls 12, 14 are insulated. As previously discussed, at least a portion of the outer wall 14 may be operably connected to an outer layer of insulation 30 and may also be operably connected to the connectors. Similarly, at least a portion of the inner wall 12 may be operably connected to an inner layer of insulation 32, including through the use of an adhesive. As with the outer layer of insulation 30, the inner layer of insulation 32 may be comprised of an insulation foam, for example a closed cell foam, or alternatively, may be comprised of multiple layers of sterile drapes that are encapsulated in a surgical grade material.

By insulating both the inner and outer walls 12, 14, the cooling medium may be able to maintain desirable temperatures during organ harvesting and/or transplantation procedures for longer periods of time so as to prevent or delay the harmful affects of warm ischemia. For example, the inner layer of insulation 32 may prevent the warming of the cooling material contained within the plurality of pillows 22 from exposure to the body heat of a patient. Additionally, the presence of the inner layer of insulation 32 may allow for the temperature of the cooling material to be lower than what may typically be achieved in embodiments that do not include an inner layer of insulation 32, such as temperatures below approximately 4° Celsius. In such circumstances, because the inner layer of insulation 32 does not function as a cooling source, but instead is an insulator, the inner layer of insulation 32 is able to insulate the cooling material from outside heat sources and ambient temperatures while also acting as a buffer against the direct exposure of the graft organ 15 to the pillows 22 and the cooled cooling material contained therein. By acting as a buffer, the inner layer of insulation 32 may prevent damage to the graft organ 15 and its tissue that may otherwise occur from direct contact with the chilled pillows 22. In such embodiments, the inner layer of insulation 32 may reach a thickness of approximately one-half an inch.

The method for using the insulation jacket 10 of the present invention includes chilling the cooling material contained within the insulation jacket 10. One factor considered in determining the appropriate chilling temperature for the cooling material is what temperatures the graft organ may be exposed to without causing cell and tissue damage. As previously discussed, the cooling material may be chilled so that when the insulation jacket 10 encloses at least a portion of the graft organ 15, the temperature of the portion of the insulation jacket 10 that comes into direct contact with the graft organ 15 should not be chilled to below approximately 4° Celsius. However, if the insulation jacket 10 includes an inner layer of insulation 32, the cooling material may be exposed to lower chilling or even freezing temperatures than what may be acceptable for embodiments of the present invention that do not include an inner layer of insulation 32.

As illustrated in FIGS. 4, 8, and 13, the insulation jacket 10 may include openings 18 that allow the insulation jacket 10 to be placed around at least a portion of the graft organ 15 before the graft organ 15 is severed from the body of the donor. Further, when harvesting a graft organ 15, because the pillows 22 may not be in communication with adjacent pillows 22, the surgeon may elect to puncture some pillows 22 in order to improve the positioning and/or enclosure of the insulation jacket 10 about at least a portion of the graft organ 15 or to improve the field of operation without destroying the cooling capabilities of insulation jacket 10.

After preparing the graft organ 15 for transplantation, at least a portion of the graft organ 15 is enclosed by the insulation jacket 10 prior to the insertion of the graft organ 15 into the body of the recipient. During this aspect of the procedure, the openings 18 provide the surgeon with access to the vessels 17 of the graft organ 15 needed for reattaching the graft organ 15 in the body of the recipient. This access permits the insulation jacket 10 to continue enclosing at least a portion of the graft organ 15 during the transplantation surgery, and thus may prevent or delay the onset of warm ischemia. As with the harvesting process, during the transplantation procedure, because of the sterile non-toxic nature of the cooling material, the surgeon also may elect to puncture some of the pillows 22 without destroying the cooling capabilities of the insulation jacket 10 or harming the graft organ 15 or patient. Upon completion of the transplantation procedure, the insulation jacket 10 may be removed from the body of the patient. Given the nature of use, the insulation jacket may be used for only one transplantation operation.

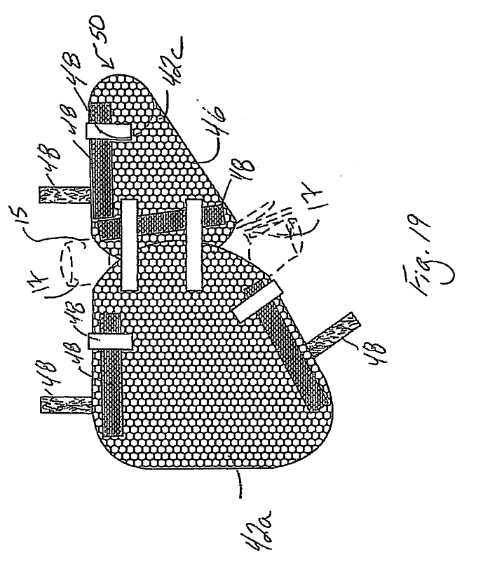

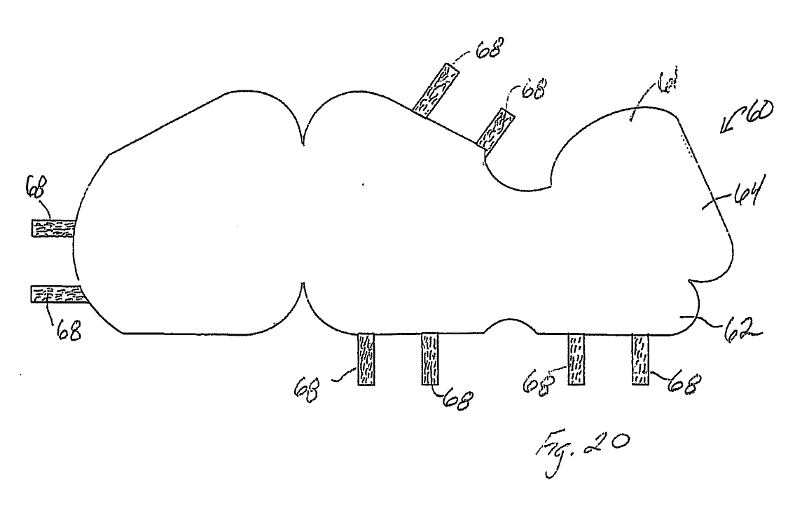

FIGS. 16 and 17 illustrate inside and outside elevation views of an insulation jacket 50 in accordance with alternative embodiment of the present invention in which insulation jacket 50 includes a body portion 40 that is comprised of an insulation foam, for example a polyethylene closed cell foam. The body portion 40 may have an inner wall 44 and an outer wall 46, as further shown in FIG. 18. The body portion 40 may also be comprised of a single panel or a plurality of panels 42a, 42b, 42c, in which case the plurality of panels 42a, 42b, 42c may be operably connected to the adjacent panel, including being connected through the use of adhesives.

FIG. 19 illustrates of an outside elevation view of an insulation jacket 50 having a plurality of panels 42a, 42b, 42c and connectors 48 in which the insulation jacket is secured about at least a portion of a graft organ in accordance with one embodiment of the present invention in which the body portion is comprised of an insulation foam. As shown, the insulation jacket 50 may be operably secured around at least a portion of the graft organ 15 through the use of connectors 48, including mating strips of hook and loop material, staples, tape, and adhesives. Additionally, the connectors 48 may be operably secured to the body portion 50, for example through the use of an adhesive material. Further, the body portion 40 may also be configured to generally conform to the shape of a specific type of a graft organ 15. By configuring the shape of the body portion 40 to generally conform to the shape of the graft organ 15, the insulation jacket 50 may have improved insulation characteristics while also minimizing the space in the operation field that is occupied by said insulation jacket 50 when the insulation jacket 50 is enclosed about at least a portion of the graft organ 15

In an alternative embodiment of the insulation jacket 50 illustrated in FIG. 19, either the inner wall 44 or the outer wall 46 of the body portion 40 may be operably connected to a liner. For example, the liner, which may be constructed from a flexible surgical grade plastic, may be connected to at least a portion of the inner wall 44 or outer wall 46 through the use of an adhesive material. When connected to the inner wall 44 or the outer wall 46 of the body portion 40, the liner may be configured to form a plurality of non-communicating pillows between said liner and the inner wall 44 or outer wall 46. The pillows may contain a cooling material that may assist in cooling or retaining the chilled temperature of a graft organ. In such an embodiment, the selection of material for the body portion 40, for example the use of a closed cell foam, may prohibit cooling material contained within the pillows from leaking or seeping through the inner or outer walls 44, 46 of the body portion 40 respectively.

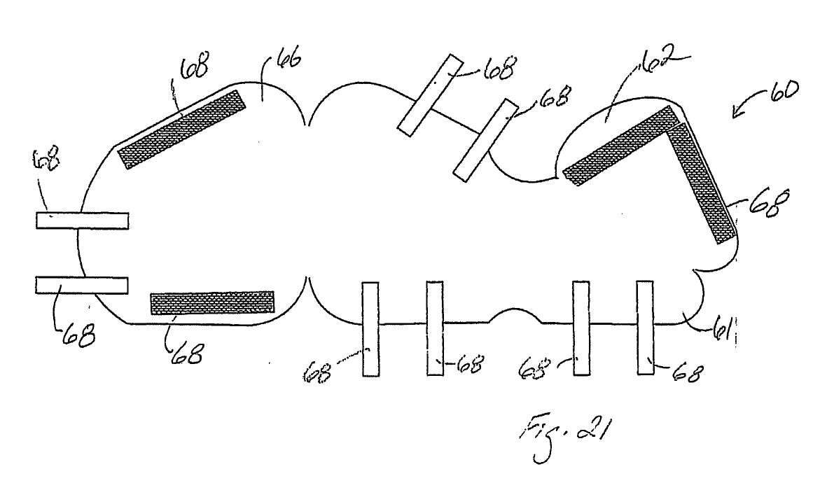

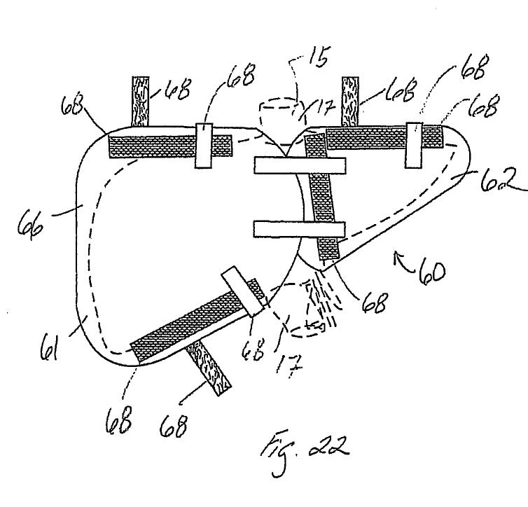

FIGS. 20 and 21 illustrate inside and outside elevation views of an insulation jacket 60 in accordance with another alternative embodiment of the present invention in which insulation jacket 60 includes a body portion 61 that is comprised of an thermo mass 62, for example a polyethylene closed cell foam. The thermo mass 62 may include an inner wall 64 and an outer wall 64 and may be comprised of, but is not limited to, a dense rubber thermo conductor gel, for example commercially available Akton™ Polymer from Action Products of Hagerstown, Md. In the illustrated embodiment, the thermo mass may be ¼ inch thick. In such an embodiment, the thermo mass 62 may have a sufficient density so that the thermo mass 62 may not have to be contained within a separate bladder, while still being sufficiently plyable so as to allow the body portion 61 to be manipulated about at least a portion of the graft organ 15.

FIG. 21 illustrates an outside elevation view of the insulation jacket 60 in which the insulation jacket 60 is secured about at least a portion of a graft organ 15. As with the previous embodiments, the thermo mass 62 may be shaped to conform to the shape of the graft organ 15 while still provide openings for the passage and/or access to the vessels 17 of the graft organ 15. Further, the body portion 61 may be operably connected to at least one connector 68, the connector 68 being configured to assist in securing the insulation jacket 60 about at least a portion of the graft organ 60. Additionally, the inner wall 64 and/or outer wall 66 of the body portion 61 may be operably connected to at least one layer of insulation, the at least one layer of insulation being configured to insulate the graft organ 15 from outside heat sources and assist in retaining the cool temperature of the chilled graft organ 15.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. An insulation jacket for thermally affecting a graft organ during harvesting and transplantation operations comprising:
a. a body portion having an inner wall and an outer wall, the inner wall operably connected to the outer wall to form a plurality of pillows, the plurality of pillows having an inner section, the plurality of pillows not being in communication with adjacent pillows, the body portion generally configured in the shape of the graft organ;
b. a cooling material located within the inner section, the cooling material being non-toxic and sterile, the cooling material contained within the plurality of pillows being capable of having a malleable nature when cooled to approximately 4 degrees Celsius; and
c. a plurality of connectors, the plurality of connectors operably connected to the insulation jacket, the plurality of connectors being configured to secure the insulation jacket about at least a portion of the graft organ.

2. The invention of claim 1 further including an outer layer of insulation, said outer layer of insulation being operably connected to the outer wall of the body portion.

3. The invention of claim 2 wherein the outer layer of insulation is comprised of a closed cell insulating foam.

4. The invention of claim 1 further including an inner layer of insulation, said inner layer of insulation being operably connected to the inner wall of the body portion.

5. The invention of claim 4 wherein the inner layer of insulation is comprised of a closed cell insulating foam.

6. The invention of claim 1 further including an outer layer of insulation and an inner layer of insulation, said outer layer of insulation being operably connected to the outer wall of the body portion, said inner layer of insulation being operably connected to the inner wall of the body portion.

7. The invention of claim 6 wherein the outer layer of insulation and the inner layer of insulation is comprised of a closed cell insulating foam.

8. The invention of claim 1, wherein the body portion has the general shape of a heart, a liver, a lung, a pancreas, or a kidney.

9. The invention of claim 1, wherein the body portion is constructed from a flexible surgical grade plastic.

10. The invention of claim 1, wherein the cooling material is a non-toxic sterile gel.

11. The invention of claim 1, wherein the cooling material is sterile saline.

12. The invention of claim 1, including a plurality of interconnecting bands positioned between the plurality of pillows, the plurality of interconnecting bands configured to assist in the ability of the insulation jacket to be manipulated about at least a portion of the graft organ.

13. The invention of claim 1 wherein the body portion is configured to form at least one opening when enclosed about at least a portion of the graft organ, said at least one opening positioned to allow at least one vessel of the graft organ to pass out of the insulation jacket.

14. The invention of claim 1 wherein the body portion is comprised of a plurality of panels.

15. The invention of claim 1, wherein the plurality of connectors includes mating strips of hook and loop material.

16. An insulation jacket for insulating a graft organ during a harvesting and transplantation operations comprising:
a. a body portion having an inner wall and an outer wall, the inner wall operably connected to the outer wall to form a plurality of non-communicating pillows and a plurality of interconnecting bands, the plurality of non-communicating pillows having an inner section, the body portion generally configured in the shape of the graft organ, the body portion being configured to form at least one opening when enclosed about at least a portion of the graft organ, said at least one opening positioned to allow at least one vessel of the graft organ to pass through the insulation jacket, the plurality of interconnecting bands positioned between the plurality of non-communicating pillows, the plurality of interconnecting bands configured to assist in the ability of the insulation jacket to be manipulated about at least a portion of the graft organ;
b. a cooling material located within the inner section, the cooling material being non-toxic and sterile, the cooling material contained within the plurality of pillows being capable of having a malleable nature when cooled to approximately 4 degrees Celsius;
c. at least one layer of insulation material, said at least one layer of insulation material being operably attached to the body portion; and
d. a plurality of connectors, the plurality of connectors operably connected to the insulation jacket, the plurality of connectors being configured to secure the insulation jacket about at least a portion of the graft organ.

17. The invention of claim 16, wherein the at least one layer of insulation material is comprised of an outer layer of insulation, said outer layer of insulation being operably connected to the outer wall of the body portion.

18. The invention of claim 16, wherein the at least one layer of insulation material is comprised of an inner layer of insulation, said inner layer of insulation being operably connected to the inner wall of the body portion.

19. The invention of claim 16, wherein the at least one layer of insulation material is comprised of an outer layer of insulation and an inner layer of insulation, said outer layer of insulation being operably connected to the outer wall of the body portion, said inner layer of insulation being operably connected to the inner wall of the body portion.

20. The invention of claim 16, wherein the body portion has the general shape of a heart, a liver, a lung, a pancreas, or a kidney.

21. The invention of claim 16, wherein the body portion is constructed from a flexible surgical grade plastic.

22. The invention of claim 16, wherein the cooling material is a non-toxic sterile gel.

23. The invention of claim 16, wherein the cooling material is sterile saline.

24. The invention of claim 16, wherein the body portion is comprised of a plurality of panels.

25. The invention of claim 16, wherein the plurality of connectors includes mating strips of hook and loop material.

26. The invention of claim 16 wherein the at least one layer of insulation is comprised of a closed cell insulating foam.

27. A method for insulating a graft organ during harvesting and transplantation operations comprising:
a. selecting an insulation jacket having the general configuration of the graft organ, the insulation jacket having a body portion, the body portion including a plurality of pillows, the plurality of pillows having an inner section, said inner section having a non-toxic sterile cooling material;
b. chilling the non-toxic sterile cooling material contained within the insulation jacket to between approximately 2° to 4° Celsius, said non-toxic sterile cooling material contained within said inner section maintaining a malleable condition when chilled to approximately 2° to 4° Celsius;
c. placing the insulation jacket inside a patient;
d. securing the insulation jacket about at least a portion of the graft organ, the insulation jacket having at least one opening configured to allow at least one vessel of the graft organ to pass through the insulation jacket and allow the surgeon access to said at least one vessel for detachment from, or reattachment to, the patient;
e. electing whether to puncture at least one of the plurality of pillows; and
f. removing the insulation jacket from the body of the patient, said removal occurring after the graft organ has been transplanted or harvested.

28. An insulation jacket for thermally affecting a graft organ during harvesting and transplantation operations comprising:
a. a body portion having an inner wall and an outer wall, the body portion generally configured in the shape of the graft organ, the body portion configured to generally conform to the shape of said graft organ when said body portion is secured about at least a portion of said graft organ, the body portion also configured to provide an opening through which at least one vessel of said graft organ may extend away from said body portion when said body portion is enclosed about at least a portion of the graft organ; and
b. a plurality of connectors, the plurality of connectors operably connected to the insulation jacket, the plurality of connectors being configured to secure the insulation jacket about at least a portion of the graft organ.

29. The invention of claim 28 wherein said body portion is comprised of a insulation foam.

30. The invention of claim 29 wherein the insulation is a closed cell foam.

31. The invention of claim 29 further including a liner, said liner and said body portion configured to form a plurality of pillows, said plurality of pillows configured to retain a cooling material, said body portion configured to prevent said cooling material from leaking through said body portion.

32. The invention of claim 28 wherein said body portion is comprised of a thermo mass.

33. The invention of claim 28 wherein said body portion is comprised of a surgical grade plastic.

34. The invention of claim 29, 32 or 33 in which the inner wall is operably connected to at least one layer of insulation.

35. The invention of claim 29, 32 or 33 in which the outer wall is operably connected to at least one layer of insulation.

36. The invention of claim 33 wherein the inner wall is operably connected to the outer wall to form a plurality of non-communicating pillows, the plurality of non-communicating pillows having an inner section, the plurality of non-communicating pillows configured to contain a cooling material, the cooling material being non-toxic and sterile, the cooling material contained within the plurality of pillows being capable of having a malleable nature when cooled to approximately 4 degrees Celsius.
